(19) United States
(12) Patent Application Publication
(10) Pub. No.: US 2012/0053633 A1
(43) Pub. Date: Mar. 1, 2012

(54) ACTUATOR FOR CORRECTING SCOLIOSIS

(75) Inventor: Roman Stauch, Assamstadt (DE)

(73) Assignee: WITTENSTEIN AG, Igersheim (DE)

(21) Appl. No.: 13/217,955

(22) Filed: Aug. 25, 2011

(30) Foreign Application Priority Data
Aug. 26, 2010 (DE) 10 2010 035 570.4
Oct. 28, 2010 (DE) 10 2010 047 738.9

(51) Int. Cl. A61F 5/00

(52) U.S. Cl. 606/237

(57) ABSTRACT

Device for the relative movement of bones, in particular for the treatment of scoliosis in humans, with a first shaft element (1), a second shaft element (2), which is connected movably to the first shaft element (1), and an electric motor, which is connected to the first shaft element (1), wherein an output shaft of the electric motor is operatively connected to the second shaft element (2) in order to permit a movement of the shaft elements (1, 2) relative to each other, wherein the electric motor (6) is arranged lengthways adjacent to at least one of the shaft elements (1, 2).

### BACKGROUND OF THE INVENTION

The invention relates to a device for the relative movement of bones, in particular to an implant for the treatment of scoliosis in humans.

Implants for treating scoliosis are known from the prior art. Scoliosis is a disease of the spinal column in which severe deformations of the spinal column occur. These deformations are generally treated mechanically, in which respect a distinction is made in principle between external treatment methods using a corset and treatment methods using implants. The treatment in each case is complex and lengthy, since the geometry of the spinal column can be changed only over a long period of time. For example, when treatment is provided with a corset, it is assumed that, if the corset is worn for up to 23 hours a day, it will take two to five years before scoliosis treatment with the corset is completed.

Treatments with implants have the advantage that the corset does not need to be worn, and therefore the treatment, at least between operations, is considered more comfortable. When providing treatment by means of implants, it is known to fuse or stiffen several vertebral bodies by using two rods. A problem, however, is that the vertebral bodies stiffen as a result of ossification or similar processes, and in some circumstances this can lead to a complete loss of mobility or to further growth being prevented.

Modern systems attempt to straighten the spinal column in steps, by means of an implanted rod system being continually adapted to the changing spinal column over the course of time. A disadvantage of this approach is that readjustment of the implant is regularly required. This necessitates surgical interventions under general anesthesia, and, in particular because of the short times between interventions, this can place a significant psychological strain on the patient.

All of the correcting methods known to date have the disadvantage that each correction has to be performed in one session, and the scope of correction is greatly limited by this. The reason for this limitation is the slow biological adaption process.

Systems comprising an implantable drive, for example as disclosed in EP 1 135 076 B1, avoid the need for frequent operations. However, for various reasons, all of the drives that have hitherto been proposed and are implantable have been used only to a limited extent in practice. In particular, it has hitherto been difficult to achieve a sufficient force with a sufficient stroke of the implant.

The object of the invention is to improve the devices known from the prior art. In particular, the object of the invention is to make available a device for the relative movement of bones, in particular an implant for the treatment of scoliosis in humans, permitting step-by-step adaptation with a sufficient stroke while reducing the number of surgical interventions.

### SUMMARY OF THE INVENTION

The object is achieved with a device according to the invention which concerns a system for scoliosis correction with a device wherein a controller is provided that is designed to control the device wirelessly or supply it inductively with energy. In particular, the device is suitable for the surgical stabilization and correction of scoliosis and deformations of the thoracic cage.

Where the word “or” is used in this application, it means, unless otherwise expressly stated to the contrary, a non-exclusive “or” in the sense of “or also”.

Advantages of the invention include, for example, fine adjustability in small steps. This leads to lower distraction and compression forces and to a greater scope of correction by possible tracking or compensation of scoliosis in the growth phase.

The device advantageously comprises a drive, in particular an electric motor. An electric motor affords the advantage of high power density. The axis of the electric motor is preferably arranged outside the two shaft elements. Moreover, the axis is preferably substantially parallel to an axis of the shaft elements. This means in particular that the axis is at least substantially parallel to a plane of curvature of the shaft elements if the latter are of a curved design. The expression “lengthways adjacent” to at least one of the shaft elements means that the electric motor is not arranged exactly in front of or behind the two elements in the longitudinal direction or direction of movement of the shaft elements. By arranging the electric motor alongside the shaft elements or alongside a direction of movement of the shaft elements, the electric motor does not unnecessarily lengthen the overall length of the device. This creates a more advantageous ratio between the possible stroke length and starting length of the overall device. Therefore, in particularly preferred embodiments of the invention, the electric motor, in the case of curved shaft elements, is arranged at least substantially perpendicular with respect to the radial plane of the curvature, laterally adjacent to the shaft elements. This affords the advantage that implantation in the human body is easier, since more room is available in this area. Moreover, this affords the advantage that, when the drive is provided via a worm wheel and a toothed rack, the engagement between worm wheel and toothed rack is obtained on an at least substantially flat area of the toothed rack. This facilitates the kinematics. In connection with the toothed rack, the expression “at least substantially flat” generally means that the surface is at least substantially uncurved, not taking into account the teeth for the drive engagement. “Substantially flat” means that the curvature on the side of engagement with the worm wheel is comparatively small, typically at most 10% or at most 20% of the curvature of the shaft element in the plane of curvature. In other typical embodiments, the surface of the toothed rack is curved, but it is less curved than the toothed rack is curved in the other direction along its longitudinal axis. In these embodiments, the toothed rack is curved in two planes. The electric motor is preferably implantable. This means that it is suitable for remaining permanently in the human body, for example in a suitable housing that advantageously encapsulates the electric motor.

In another aspect of the invention, an actuator composed of shape-memory alloy or of a piezo element is used as drive. Like the electric motor, it is arranged laterally alongside the shaft elements. The comments that have been made in this application with reference to the electric motor, in particular to the arrangement thereof, apply analogously to the actuator. Likewise, preferred features that are described below for a device with an electric motor can also advantageously be used in a device with an actuator composed of a shape-memory alloy or of a piezo element. Shape-memory alloys afford the advantage that they can be used in a small space and are advantageously completely biocompatible. Piezo elements afford the advantage of being very small and taking up little space.

The position of the electric motor is preferably lengthways adjacent to the shaft elements, which, in the case of curved shaft elements, preferably means that the axis of the electric motor is parallel to a plane of curvature of the shaft elements and shifted perpendicular thereto until outside the shaft elements. The electric motor preferably comprises a stator and a rotor, which are each equipped with electromagnets or permanent magnets.

The shaft elements are connected to each other and are movable relative to each other. Preferably, the shaft elements are movable telescopically one inside the other. This affords the advantage of providing a particularly stiff connection of the shaft elements while at the same time allowing their movement. The shaft elements can have different profile shapes, for example with hollow profiles having the advantage of being particularly stiff while having low weight and using up little material. Another possibility is to use a T-profile for one of the shaft elements, in which case the T-profile can be moved in a C-profile that forms the other shaft element. Tubular shaft elements are particularly preferable, in which case the cross section of the tube preferably corresponds to a rectangle or square, each advantageously rounded. As regards the geometry of the cross section of the shaft elements, other curvatures are also possible.

A gear, preferably with a transmission ratio, is preferably arranged in the operative connection between the electric motor and the second shaft element. Thus, the gear preferably has a transmission ratio of at least 1:250, preferably of at least 1:1000, preferably of at least 1:2000, and more preferably of at least 1:4000. The gear permits the use of a comparatively small motor with a low torque, while still achieving a sufficient force between the standard fastenings of the shaft elements. This greatly influences the implantability, since only a small motor has to be used that can also be supplied with energy electromagnetically by induction.

The gear is preferably designed as a planetary gear or comprises a planetary gear. Planetary gears afford the advantage of a particularly large transmission ratio within a small space. Moreover, the planetary gear preferably has a diameter of less than 20 mm, preferably less than 15 mm or less than 12 mm. Likewise, a motor preferably has a diameter of at most 20 mm, preferably at most 15 mm, more preferably at most 12 mm. Motor and gear are preferably arranged axially one behind the other, such that they form a unit. This affords the advantage of a compact construction. In interaction with the gear, the electric motor achieves, with inclusion of a force transmission to the toothed rack, a force of at least 100 N, preferably at least 300 N, between the shaft elements. Such a force is sufficient to permit treatment of scoliosis.

It is preferably possible to lengthen the shaft elements by at least 40%, preferably by at least 50% and more preferably by at least 80% of their initial length. The length is typically measured between two bone fasteners. The bone fasteners are each secured on the shaft elements and preferably permit a connection of the shaft elements in each case to a vertebra or a costal arch. Advantageously, the shaft elements are movable relative to each other by at least 30 mm, preferably at least 40 mm. This permits longer-lasting treatment, without the need for an invasive intervention on the patient betweentimes.

The electric motor is preferably arranged in a housing, which is pushed laterally onto the first shaft. The housing is advantageously fixed on the first shaft by at least one screw, preferably at least two screws. Once again, laterally means that the housing is pushed on in the axial direction with respect to the curvature, that is to say in a direction at least substantially perpendicular to the plane of curvature. This affords the advantage that the electric motor, if appropriate integrated with the gear in the housing, can be exchanged as a modular element. The lateral pushing-on affords the advantage that, laterally with respect to the shaft elements in the human body, there is space for such a drive. The electric motor is preferably arranged, together with the gear and, if appropriate, a driving toothed wheel, for example a pinion, a thread or a spindle, in the housing that is pushed laterally onto the first shaft. This affords the advantage of a compact construction. In typical embodiments, the housing is integral with the first shaft element. In this case, the motor is preferably arranged lengthways adjacent to the second shaft element or to a direction of travel of the second shaft element. Here, “integral” means in particular that the housing is connected fixedly to the first shaft or is integrated therein.

A direct-current motor or a stepping motor is preferably used as electric motor. Direct-current motors afford the advantage of high power density and smooth running. Stepping motors afford the advantage of exact adjustability of the movement of the shaft elements relative to each other. Advantageous embodiments of the invention are unbraked. The term “unbraked” means that the device has no brake or catch, in which respect it should be noted that, particularly with the aid of a stepping motor in combination with the gear transmission ratio, a sufficient self-locking is achieved in order to prevent inadvertent movement of the shaft elements relative to each other. However, it is also possible in embodiments of the invention to provide a brake or catch, particularly in conjunction with a simple direct-current motor. A brake affords the advantage of reliable fixing. An unbraked system affords the advantage of a simple and compact construction and of controlled forward and backward running.

In preferred embodiments, the second shaft element comprises a toothed rack. The toothed rack is preferably integral with the second shaft element or forms the second shaft element. The toothed rack is preferably driven by a worm wheel. The toothed rack affords the advantage of permitting a stroke that is limited only by the length of the first shaft element.

At least one of the shaft elements preferably has a curvature of at most 300 mm radius. Particularly preferably, the curvature is of at most 220 mm radius or 70 mm radius. The toothed rack is also advantageously curved, in particular at least substantially in the same radius as at least one of the shaft elements. Particularly preferably, both shaft elements and the toothed rack are provided at least substantially with an identical curvature. Other embodiments of the invention have at least in part a straight toothed rack or a straight spindle for an engagement with a drive element, such as a worm or a pinion of the gear.

Preferred embodiments of the invention have a pinion arranged on the output side of the gear and in engagement with a toothed wheel. The toothed wheel has an internal thread, which is in engagement with a spindle. The spindle is preferably part of the second shaft element or forms the second shaft element. In typical embodiments of the invention, additional spur gears can be interposed in order to reduce an overall width of the device.

The toothed rack or the spindle is preferably connected to the second shaft element and, during movement of the two shaft elements relative to each other, moves inside the first shaft element. For engagement with a driving toothed wheel, the first shaft element preferably has an opening, particularly preferably a lateral opening on the lengthways side on which the electric motor is arranged. The curvature of the shaft elements affords the advantage that the scoliosis correction can be performed in an anatomically advantageous manner. The lateral opening ensures that the toothed rack is arranged in a protected manner inside the first shaft element, and the lateral opening in the first shaft element is preferably closed by the drive housing comprising the electric motor and the gear when the drive is pushed on.

A worm meshing with the toothed rack is preferably arranged on the output side of the gear. The worm preferably has a spindle pitch of more than 0.3 mm, preferably of more than 0.5 mm, or of less than 1.5 mm, preferably of less than 1 mm. The worm, which is driven by the electric motor, if appropriate with interposed gear, is preferably in engagement with the toothed rack on one of the uncurved surfaces of the toothed rack. The toothed rack is advantageously curved in exactly one direction of its rectangular cross section. In typical embodiments, rectangular shaft elements are curved in exactly one direction or one plane. The toothed rack thus has two surfaces which are not curved or extend only in the plane of their surface “about the curve”. Such a curve of the flat surface is unproblematic for an engagement of the worm in the teeth of the toothed rack.

The device preferably comprises a control means, which is connected to the electric motor and is suitable for receiving energy or control signals wirelessly. The control means preferably comprises a receiver unit and electronic components. The control means is preferably implantable, in particular can be arranged subcutaneously, and is intended and suitable for remaining permanently in the human body during treatment of scoliosis. In this way, it is possible to control the drive and supply energy to it without another operation being needed. The energy transmission is preferably inductive. For this purpose, the control means preferably comprises an electromagnetic coil (magnet coil). Preferred embodiments of the control means are without energy storage. This affords the advantage of a simple construction. It has been found that an energy storage can be dispensed with in some circumstances. By contrast, typical embodiments of the invention do have energy storages, in order to reduce the intensity of the electromagnetic radiation during operation of the device.

Between the electric motor and the control means there is preferably a feed line or cable connection with an electrically conductive connection or an electrically conductive cable. The control means is preferably designed to control or regulate the drive and preferably comprises an integrated switching circuit in order to perform appropriate functions. This affords the advantage of a compact structure with great functionality.

Preferred embodiments of the control means comprise a return channel, such that it is possible to transmit information from the device or from the control means, when these are implanted, to an external controller. The control means is preferably configured for bidirectional data transmission.

The control means is preferably designed to control the drive in such a way that it is possible for the shaft elements to be driven apart and driven one inside the other. This affords the advantage that reverse travel is also possible, for example if too great a force is determined. Preferably, the electric motor, the gear or other parts of the device are likewise suitably configured to allow the two shaft elements to move forward and backward relative to each other.

The device preferably comprises a sensor, which is arranged in order to detect a relative movement or a force between the shaft elements. The control means is preferably suitable for processing or transmitting information from the sensor, preferably to a controller arranged outside the body. Moreover, the control means preferably comprises a regulating unit which, on the basis of the sensor signal, controls the electric motor in order to reach a predefined force value or movement value. It is likewise possible to establish the control loop via an external controller. This affords the advantage of an exact adjustment.

The controller, which is arranged outside the body, preferably comprises an electromagnetic coil in order to transmit energy and signals to the control means inductively. The controller is preferably configured to control the device wirelessly via the control means.

### BRIEF DESCRIPTION OF THE DRAWINGS

Preferred illustrative embodiments of the invention are explained with reference to the attached drawings, in which:

FIG. 1 shows a device according to the invention in a schematic and partially sectioned plan view;

FIG. 2 shows a perspective view of part of the device according to the invention from FIG. 1;

FIG. 3 shows a schematic and partially sectioned view of part of the device from FIG. 1; and

FIG. 4 shows a schematic and partially sectioned view of another embodiment of a device according to the invention for treatment of scoliosis together with a controller as a system for treating scoliosis.

### DETAILED DESCRIPTION

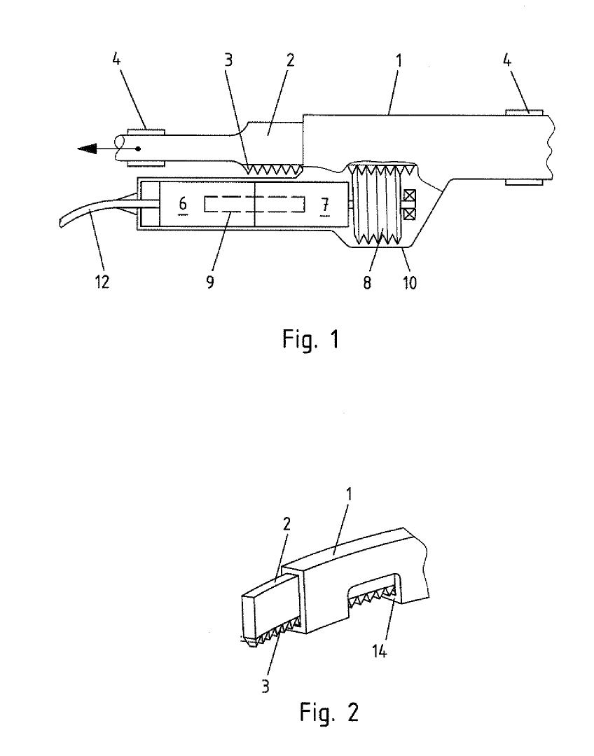

FIG. 1 shows a preferred embodiment of a device according to the invention in a schematic and partially sectioned view.

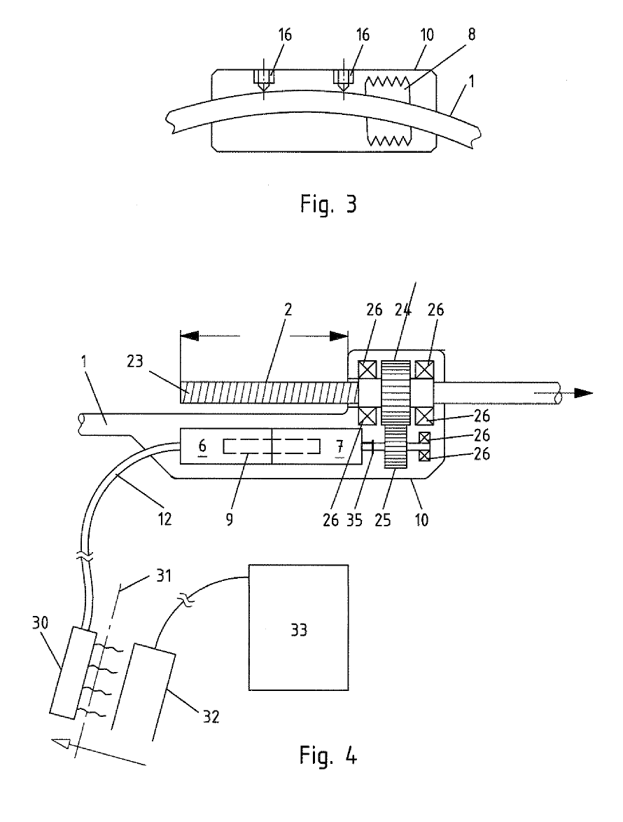

The device according to the invention in FIG. 1 comprises a first shaft element 1 and a second shaft element 2. The second shaft element 2 integrally comprises a toothed rack 3. The toothed rack 3 forms the second shaft element 2. The respective ends of the shaft elements 1 and 2 are provided with bone fasteners 4, which are designed to engage in costal arches. In the direction of an arrow shown in FIG. 1, the second shaft element 2 is movable relative to the first shaft element 1 in both senses, i.e. bidirectionally. To obtain such a movement, an electric motor 6 is used, which drives a worm wheel 8 via a planetary gear 7 with a transmission ratio. The worm wheel 8 is driven via an output shaft 9 (shown by broken lines) of the electric motor 6 and via the planetary gear 7. The electric motor 6, the planetary gear 7 and the worm wheel 8 are arranged in a housing 10, which is pushed laterally onto the first shaft element 1 and is fixed on the first shaft element 1. The securing of the housing 10 on the first shaft element 1 is shown in more detail in FIG. 3.

The device shown in FIG. 1 can advantageously be used for scoliosis treatment, wherein the two bone fasteners 4 are connected to costal arches or vertebral bodies. Driving the shaft elements 1 and 2 apart has the effect of straightening a spinal column. The shaft elements 1 and 2 can be driven apart in steps over a long period of time without surgical interventions being carried out betweentimes.

The advantage of the device shown is that it is completely implantable. The electric motor 6 is connected via a feed line 12 or a cable to a control means (not shown in FIG. 1) likewise provided for implantation. The control means comprises electronics and a receiver, likewise remains in the body, and is able to inductively receive and evaluate control signals and energy for controlling and powering the electric motor 6. This is explained in more detail in connection with the illustrative embodiment in FIG. 4.

By virtue of the high transmission ratio of the planetary gear 7 and the transmission between the toothed rack 3 and the worm wheel 8, a high degree of self-locking between the electric motor 6 and the driven second shaft element 2 is achieved, such that a brake or a catch is not necessarily required in order to ensure, without supply of energy, that the shaft elements 1 and 2 are held in a position to which they have been driven. In this way, a very simple system is created which additionally allows the second shaft element 2 to be driven forward and backward relative to the first shaft element 1.

The shaft elements 1 and 2 of the device shown in FIG. 1 are curved, with the plane of curvature perpendicular to the drawing plane. This means that the surface of the toothed rack 3 on which the teeth of the toothed rack 3 are arranged is uncurved or flat. Here, “flat” again means with the exception of the elevations of the teeth of the toothed rack 3. The advantage of the arrangement shown in FIG. 1, with the described exemplary curvature, is that the curvature of 220 mm radius can be advantageously used for scoliosis treatment. Other preferred embodiments have a curvature of 70 mm radius. At the same time, an arrangement of the electric motor on the depicted lengthwise side of the shaft elements 1 and 2 is advantageous, since it is precisely at this location that space is available from the medical point of view.

Details of the shaft elements 1 and 2 from FIG. 1 are shown once again in FIG. 2. In the description of FIGS. 2 to 4, the same reference signs are used for the same or similar parts.

The curvature of the shaft elements 1 and 2 can be seen in FIG. 2, where the rectangular, solid second shaft element 2 is guided in the rectangular tube of the first shaft element 1. FIG. 2 also shows an opening 14 provided for the engagement of the worm wheel 8 in the toothed rack 3.

In typical embodiments, the second shaft element is likewise hollow or of tubular design, and therefore the second shaft element can be designed as a hollow toothed rack or spindle. These afford the advantage of a compact and light construction. In other typical embodiments, force is transmitted via a pinion or a thread.

FIG. 3 shows how the housing 10 is pushed onto the shaft element 1. The cross-sectional view shows how the housing 10 can be fixed on the shaft element 1 with two screws 16. In FIG. 1, the shaft element 1 is shown partially in cross section, and the housing 10 in FIG. 1 is also shown partially in cross section, in order to show the worm wheel 8, the motor 6 and the gear 7.

FIG. 4 shows another embodiment according to the invention, with FIG. 4 also showing in particular the control means 30, which is likewise advantageously provided in the device in FIG. 1. Once again, the same reference signs are used for the same or similar parts.

The embodiment in FIG. 4 differs from the embodiment in FIG. 1 firstly in two ways. The electric motor 6 and the gear 7 are arranged to the side of the shaft element 2, but the housing 10 in which the electric motor 6 and the planetary gear 7 are arranged is integrated with the first shaft element 1. Transmission of force from the output shaft 9 to the shaft element 2 is possible via a spindle 23, which is part of the shaft element 2. The spindle 23 is in engagement with an internal thread of a toothed wheel 24, which is driven by a pinion 25. The pinion 25 is driven by the electric motor via the planetary gear 7. Bearings 26 are provided for mounting the shaft element 2 on the housing 10 designed integrally with the shaft element 1 and for mounting the pinion.

The electric motor 6 is connected via the feed line 12 to the control means 30, which is supplied inductively with energy and control signals. The control means 30 is intended to be implanted in the human body, in particular under the skin 31 of the human body. In order to emit energy and control signals and to receive return channel data, i.e. for bidirectional data exchange, a transmission unit 32 is used, which is connected to a control unit 33. The control unit 33 and the transmission unit 32 together form a controller with which the device, which comprises the elements of the invention arranged inside the body, can be controlled wirelessly via the control means 30 and can be supplied inductively with energy. It is thus possible, by way of the control means 30, for the control unit 33 to control the electric motor 6, which is designed as stepping motor, in such a way that a defined stroke of the shaft elements 1 and 2 relative to each other is achieved.

By way of a sensor 35, which is arranged on the planetary gear output shaft, a torque load on this output shaft can be tested and, in this way, it is possible to deduce the force that exists between the shaft elements 1 and 2. This force is transmitted by the control means 30 to the transmission unit 32 via a return channel and onward to the control unit 33. If the force is too great, the control unit 33 can stop an advance movement of the electric motor 6 by means of the above-described control via the control means 30. In this way, a control loop can be set up.

In advantageous embodiments of the invention, a sensor is arranged under one of the bearings 26 and can likewise be used to determine the force that exists between the shaft elements. This affords the advantage of a simple construction.

In typical embodiments of the invention, the housing in which the electric motor and the planetary gear are arranged is designed integrally with the first shaft element. This affords the advantage of a simple construction. The electric motor is then arranged lengthways adjacent to the second shaft element. In this application, “lengthways adjacent” generally means that the electric motor is arranged lengthways adjacent to an axis or an extension of the first or second shaft element, even when the corresponding shaft element is no longer exactly laterally next to the electric motor during operation but is moved as a result of the device being driven apart.

In other embodiments of the invention, the sensor can also measure a distance or a rotation and, by means of the return channel, transmit these to the controller via the control means, such that, if a simple direct-current motor without stepping motor properties is used, a control loop can be established in order to control defined stroke lengths of the shaft elements.

The invention is not limited to the preferred embodiments described above. The scope of the invention is defined instead by the claims.

1. Device for the relative movement of bones for the treatment of scoliosis in humans, comprising:
a first shaft element (1);
a second shaft element (2), movably connected to the first shaft element (1); and
an electric motor provided with a housing which is secured to the first shaft element (1), the electric motor has an output shaft operatively connected to the second shaft element (2) in order to permit a movement of the first and second shaft elements (1, 2) relative to each other, wherein the electric motor (6) is arranged lengthways adjacent to at least one of the shaft elements (1, 2).

2. Device according to claim 1, wherein a gear arrangement comprises the operative connection between the output shaft and the second shaft element (2).

3. Device according to claim 2, wherein the gear arrangement has a transmission ratio of at least 1:250.

4. Device according to claim 1, wherein the gear arrangement comprises a planetary gear (7).

5. Device according to claim 1, wherein the electric motor is arranged in the housing, wherein the housing is pushed laterally and releasably onto a lengthwise side of the first shaft element (1).

6. Device according to claim 1, wherein the first and second shaft elements (1, 2) are movable telescopically one inside the other.

7. Device according to claim 1, wherein at least one of the first and second shaft elements (1, 2) has a curvature of at most 300 mm radius.

8. Device according to claim 1, wherein a control means (30) for receiving energy and/or control signals wirelessly is connected to the electric motor.

9. Device according to claim 8, wherein the control means (30) controls the electric motor (6) in such a way that it is possible for the first and second shaft elements (1, 2) to be driven apart and to be driven one inside the other.

10. Device according to claim 1, wherein a sensor (35) is provided to detect a relative movement and/or a force between the first and second shaft elements (1, 2).

11. Device according to claim 1, wherein the second shaft element (2) comprises a toothed rack (3) which is in engagement with a worm wheel (8) driven by the electric motor (6).

12. Device according to claim 11, wherein the worm wheel (8) engages in teeth of the toothed rack (3) on an uncurved surface of the toothed rack (3).

13. Device according to claim 1, wherein the second shaft element (2) comprises a spindle (23) engaging a toothed wheel (24) driven by the electric motor (6), the toothed wheel (24) has an internal thread.

14. Device for the relative movement of bones for the treatment of scoliosis in humans, comprising:
a first shaft element (1),
a second shaft element (2) movably connected to the first shaft element (1); and
an actuator, which is arranged lengthways adjacent to at least one of the first and second shaft elements (1, 2) and is connected to the first shaft element (1), the actuator is provided with an output element operatively connected to the second shaft element (2) in order to permit a movement of the first and second shaft elements (1, 2) relative to each other, wherein the second shaft element (2) comprises a toothed rack (3).

15. System for correcting scoliosis with a device according to claim 1 for implantation in a human body, wherein a controller (32, 33) is designed to control the device wirelessly via the control means (30) and to supply it inductively with energy.
